(19) United States
(12) Patent Application Publication
(10) Pub. No.: US 2019/0110703 A1
(43) Pub. Date: Apr. 18, 2019

(54) PULSE DIAGNOSIS APPARATUS AND PULSE DIAGNOSIS METHOD THEREOF

(71) Applicants: Kwangjae LEE, Seoul (KR); Sukbong KWON, Seoul (KR)

(72) Inventors: Kwangjae LEE, Seoul (KR); Sukbong KWON, Seoul (KR)

(21) Appl. No.: 15/798,915

(22) Filed: Oct. 31, 2017

(30) Foreign Application Priority Data
Oct. 12, 2017 (KR) 10-2017-0132300

(51) Int. Cl. A61B 5/024; A61B 5/11; A61B 5/00; A61B 5/021

(52) U.S. Cl. CPC A61B 5/02438; A61B 5/11; A61B 5/681; A61B 5/222; A61B 5/6804; A61B 5/6831; A61B 5/021

(57) ABSTRACT

The present invention relates to a pulse diagnosis apparatus, and more particularly, to a pulse diagnosis apparatus which includes a blood flow rate sensor, continuously observes a blood flow rate change of a user, and diagnoses a pulse of the user by using a pulse model generated by using a deep neural network (DNN) and a pulse diagnosis method used in the pulse diagnosis apparatus.

### CROSS-REFERENCE TO RELATED APPLICATION

This application claims priority to and the benefit of Korean Patent Application No. 10-2017-0132300 filed in the Korean Intellectual Property Office on Oct. 12, 2017, the entire contents of which are incorporated herein by reference.

The present invention relates to a pulse diagnosis apparatus, and more particularly, to a pulse diagnosis apparatus which includes a blood flow rate sensor, continuously observes a blood flow rate change of a user, and diagnoses a pulse of the user by using a pulse model generated by using a deep neural network (DNN), and a pulse diagnosis method used in the pulse diagnosis apparatus.

The content described herein merely provides background information with respect to embodiments, and does not configure related art.

A wearable device is a device worn on a user's body and capable of performing a computing operation, and may be embodied in a variety of types of wearable devices such as a watch, glasses, or the like which are wearable on the user's body.

The wearable device is worn on the user's body and collects movement data of the user, and accordingly calculates the number of steps the user takes. In addition, various pieces of health information such as an amount of exercise and a heart rate may be monitored and provided to the user. Accordingly, a demand for wearable devices has been gradually increasing.

However, since existing wearable devices simply check a heart rate or the like and notify a user of the checked heart rate in a digitized form, it is difficult to perform a more detailed diagnosis.

Meanwhile, as the quality of life has been improved and medical technologies have been developed, people are increasingly interested in health and medical treatment. Particularly, chronic diseases including high blood pressure, cerebrovascular diseases, and heart diseases may be diagnosed in their early stages by monitoring a pulse measurable at a human body and determining whether there is a change to the pulse. Particularly, the pulse is a phenomenon in which arteries are dilated and constricted according to resistance transferred to blood vessels when a heart releases blood, and shows a periodic waveform shape. Accordingly, it is necessary to develop a pulse diagnosis apparatus capable of analyzing a waveform of a pulse, diagnosing a condition of a user's health, and prescribing treatment.

However, since existing pulse diagnosis apparatuses are provided in medical institutions such as hospitals and clinics such that a pulse state of a visiting user is diagnosed by checking only fragmentary pulse information of the user, it is difficult to continuously check a pulse of the user. Also, since the existing pulse diagnosis apparatuses simply output a waveform of a pulse of a user to allow a specialist such as a medical doctor to diagnose the pulse by analyzing a meaning of the waveform, the diagnosis depends on experience of the specialist and there are limitations in a lack of objectivity and reproducibility.

### PRIOR ART DOCUMENT

### Patent Document

Korean Patent Registration No. 10-1770040, published on Aug. 14, 2017 (titled FITNESS TRAINING METHOD BASED ON WEARABLE DEVICE, FITNESS TRAINING SYSTEM AND WEARABLE DEVICE FOR FITNESS TRAINING)

The present invention is directed to providing a pulse diagnosis apparatus capable of being worn on a user's body part and diagnosing a pulse of the user by continuously observing a blood flow rate change of the user, and a pulse diagnosis method used in the pulse diagnosis apparatus.

The present invention is also directed to providing a pulse diagnosis apparatus capable of continuously observing a blood flow rate change of a user, diagnosing a pulse by using a learning model generated using a deep neural network (DNN), building a learning model customized for the user by continuously learning using the pulse diagnosis result of the user, and more accurately diagnosing the pulse by using the learning model, and a pulse diagnosis method used in the pulse diagnosis apparatus.

However, aspects of the present invention are not limited to the above-described aspects, and additional unstated aspects of the present invention will be obvious from the following description.

One aspect of the present invention provides a pulse diagnosis apparatus including a sensor portion which includes a blood flow rate sensor capable of measuring a blood flow rate change and generates a blood flow rate waveform through the blood flow rate sensor, an apparatus controller which controls an application of the generated blood flow rate waveform to a pulse model generated using a DNN, and a pulse diagnosis portion which derives pulse diagnosis data with respect to the blood flow rate waveform by using the pulse model.

The sensor portion may further include a pressure sensor, and the apparatus controller may control the pressure sensor to generate a certain level of pressure when a preset condition occurs.

The apparatus controller may control an application of a blood flow rate waveform changed according to a pressure change generated by the pressure sensor and a pressure value generated by the pressure sensor to the pulse model.

When the pressure sensor includes a pressure generation module which generates the pressure and a measurement module which measures a vibration of a pulse according to the pressure generated by the pressure generation module and generates a pulse waveform, and the apparatus controller may control an application of a blood flow rate waveform changed according to a pressure change generated by the pressure generation module and a pulse waveform generated by the measurement module to the pulse model.

The apparatus controller may monitor the blood flow rate waveform changed according to the pressure change or may check a level of the vibration measured by the measurement module to adjust a position to which the pressure is applied.

The pulse diagnosis apparatus may further a diagnosis learning portion which builds the pulse model through pulse model training using a DNN based on previously obtained clinical outcome data and builds a medical examination model through medical examination model training using a recurrent neural network (RNN).

Another aspect of the present invention provides a pulse diagnosis apparatus including a sensor portion which includes a blood flow rate sensor capable of measuring a blood flow rate change and generates a blood flow rate waveform through the blood flow rate sensor, a diagnosis learning portion which learns and generates a pulse model by applying the generated blood flow rate waveform set to be an input and pulse data set to be an output to a DNN, and a pulse diagnosis portion which performs a pulse diagnosis with respect to a blood flow rate waveform periodically measured through the sensor portion by applying the blood flow rate waveform to the pulse model.

The diagnosis learning portion may learn and generate a medical examination model through medical examination model training by applying basic diagnosis data including at least one of medical examination data generated through a question and answer of a user and prestored diagnosis history data to an RNN, and the pulse diagnosis portion may derive pulse diagnosis data by applying pulse data derived through the pulse model and the medical examination data derived through the medical examination model to the DNN as an input thereof.

Another aspect of the present invention provides a pulse diagnosis method including generating a blood flow rate waveform through a blood flow rate sensor capable of measuring a blood flow rate change and deriving, by a pulse diagnosis apparatus, pulse data with respect to the measured blood flow rate waveform by applying the blood flow rate waveform to a pulse model generated using a DNN.

When the pulse diagnosis apparatus further includes a pressure sensor, the generating of the blood flow rate waveform may include monitoring the measured blood flow rate waveform. controlling the pressure sensor to generate a certain level of pressure when it is determined that a preset condition occurs as a result of the monitoring, and continuously measuring and generating a blood flow rate waveform changed according to a pressure change.

The pulse diagnosis method may include, when the pressure sensor includes a pressure generation module which generates the pressure and a measurement module which measures a vibration of a pulse according to the pressure generated by the pressure generation module and generates a pulse waveform, generating a blood flow rate waveform changed according to the pressure change and the pulse waveform by using the measurement module.

The pulse diagnosis method may further include, when basic diagnosis data which includes at least one of medical examination data generated through a question and answer of a user and prestored diagnosis history data is checked, deriving medical examination data with respect to the basic diagnosis data by applying the basic diagnosis data to a medical examination model generated using an RNN.

The pulse diagnosis method may further include, after the deriving of the medical examination data, deriving pulse diagnosis data through an RNN which sets the pulse data and the medical examination data to be an input value and sets the pulse diagnosis data to be an output value.

Another aspect of the present invention provides a computer-readable recording medium in which a program for executing the method disclosed above is recorded.

According to a pulse diagnosis apparatus and a pulse diagnosis method used in the pulse diagnosis apparatus, a pulse diagnosis apparatus capable of being worn on a user's body part may continuously observe a blood flow rate change of the user and diagnose a pulse according to the blood flow rate change.

Also, according to the present invention, a pulse of a user may be diagnosed by using the pulse diagnosis apparatus continuously worn on a part of the user's body without additionally visiting a medical institution such as a clinic, and a change in body conditions of the user may be immediately checked through the diagnosis.

Also, according to the present invention, a learning model customized for a user may be built by continuously observing a blood flow rate change of the user, diagnosing a pulse by using a learning model generated by a DNN, and continuously learning using the pulse diagnosis result of the user such that it is possible to more accurately perform a pulse diagnosis through the customized learning model.

Also, according to the present invention, there are provided advantages in that pulse data is derived by using a DNN, medical examination data is derived by using a RNN, and final pulse diagnosis data is derived by using the DNN again such that a neural network algorithm adequate for a pulse diagnosis may be built and the pulse diagnosis may be more accurately performed.

In addition, a variety of effects in addition to the above-described effects may be directly or indirectly disclosed in the detailed description for embodiments of the present invention which will be described below.

### BRIEF DESCRIPTION OF DRAWINGS

FIG. 1 is a schematic diagram of a pulse diagnosis system according to one embodiment of the present invention.

FIGS. 2A and 2B are views illustrating an example of a pulse diagnosis apparatus according to one embodiment of the present invention.

FIGS. 3A and 3B are views illustrating a state in which the pulse diagnosis apparatus according to one embodiment of the present invention is worn on a part of a user's body.

FIG. 4 is a configuration diagram illustrating significant components of the pulse diagnosis apparatus according to one embodiment of the present invention.

FIGS. 5A, 5B and 5C are views illustrating an example of a waveform according to one embodiment of the present invention.

FIG. 6 is a view illustrating an example of a process of building a learning model according to one embodiment of the present invention.

FIG. 7 is a view illustrating an example of a deep neural network (DNN) for building a learning model according to one embodiment of the present invention.

FIG. 8 is a view illustrating a process of deriving pulse diagnosis data by using a neural network algorithm according to one embodiment of the present invention.

FIG. 9 is a schematic flowchart illustrating a pulse diagnosis method according to one embodiment of the present invention.

FIG. 10 is a more detailed flowchart illustrating the pulse diagnosis method according to one embodiment of the present invention.

### DETAILED DESCRIPTION

Exemplary embodiments of the present invention will be described in detail with reference to the attached drawings to clearly describe features and advantages of limitation-overcoming means of the present invention.

In the following description, detailed descriptions of well-known functions or constructions will be omitted since they would obscure the invention with unnecessary detail. Also, it should be noted that throughout the drawings, like reference numerals refer to like elements.

It should be understood that the terms used in the specification and the appended claims are not to be construed as limited to general and dictionary meanings but should be interpreted based on the meanings and concepts corresponding to technical aspects of the present invention on the basis of the principle that the inventor is allowed to define terms appropriately for the best explanation. Therefore, the description proposed herein is just a preferable example for the purpose of illustrations only and is not intended to limit the scope of the invention, and thus it should be understood that other equivalents and modifications can be made thereto without departing from the spirit and scope of the invention.

Although the terms such as first, second, and the like may be used to describe various elements, the elements are not limited by the terms. The terms are used only for distinguishing one element from other elements. For example, without departing from the scope of the present invention, a second component may be referred to as a first component, and similarly, a first component may be referred to as a second component.

In addition, when it is stated that one component is “coupled” or “connected” to another component, the one component may be logically or physically coupled or connected to the other component. In other words, although one component may be directly coupled or connected to another component, it should be understood that yet another component may be present therebetween or that the two components are indirectly coupled or connected to each other.

Terms are used herein only to describe particular embodiments and are not intended to limit the present invention. Singular forms, unless defined otherwise in context, include plural forms. Throughout the specification, it should be understood that the terms “comprise,” “have,” and the like are used herein to specify the presence of stated features, numbers, steps, operations, elements, components, or combinations thereof, but do not preclude the presence or addition of one or more other features, numbers, steps, operations, elements, components, or combinations thereof.

Additionally, embodiments within the scope of the present invention may include a computer-readable medium which has or transfers a computer-executable instruction or a data structure stored in the computer-readable medium. The computer-readable medium may be a random usable medium accessible by a universal or special-purpose computer system. For example, the computer-readable medium may include a random-access memory (RAM), a read-only memory (ROM), an erasable programmable ROM (EPROM), a compact disc ROM (CD-ROM), other optical disk storage devices, a magnetic disk storage device, other magnetic storage devices, and a physical storage medium such as other random media accessible by the universal or special-purpose computer system, and may be used to store or transfer a computer-executable instruction, a computer-readable instruction, or a predetermined program code means having a data structure, but the computer-readable medium is not limited thereto.

In the following description and claims, “network” or “communication network” are defined to be one or more data links capable of transmitting electronic data among computer systems and/or modules. When data is transmitted or provided to a computer system through a network or other (wired, wireless, or a wired/wireless-combined) communication interface, the connection may be understood as a computer-readable medium. A computer-readable instruction, for example, includes an instruction and data which allows a universal computer system or a special-purpose computer system to perform a particular function or functional group. A computer-executable instruction may be, for example, an assembly language or even binary, and an intermediate format instruction such as a source code.

In addition, the present invention may be executed in a network computing environment having a variety of types of computer system configurations which includes a personal computer (PC), a laptop computer, a handheld device, a multiprocessor system, microprocessor-based or programmable consumer electronics, a network PC, a mini computer, a main frame computer, a mobile telephone, a personal digital assistant (PDA), a pager, and the like.

The present invention may be executed in a distributed system environment in which all local and remote computer systems linked by a combination of a wired data link, a wireless data link, and a wired and wireless data link perform a task through a network. In the distributed system environment, a program module may be located in a local memory storage device and a remote memory storage device.

Hereinafter, significant components of a pulse diagnosis system according to one embodiment of the present invention will be described.

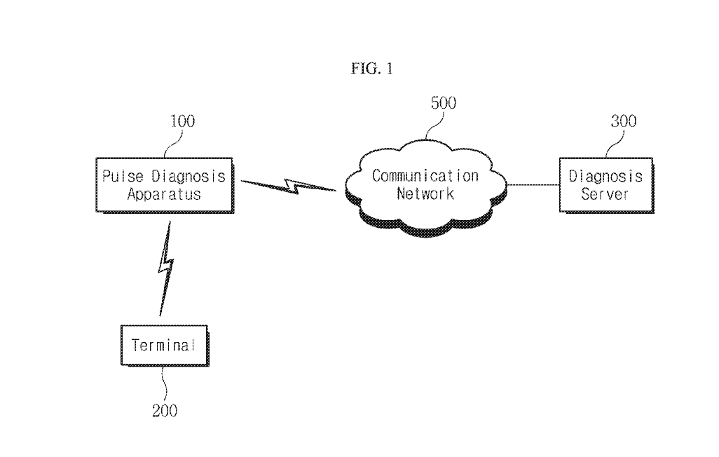

FIG. 1 is a schematic diagram of a pulse diagnosis system according to one embodiment of the present invention.

Referring to FIG. 1, the pulse diagnosis system according to one embodiment of the present invention may include a pulse diagnosis apparatus 100 capable of continuously diagnosing a pulse of a user.

The pulse diagnosis apparatus 100 according to one embodiment of the present invention refers to an apparatus configured to include a blood flow rate sensor and capable of continuously measuring a blood flow rate change by using the blood flow rate sensor. Also, the pulse diagnosis apparatus 100 may derive pulse diagnosis data of a waveform of a blood flow rate measured by using a pulse model built using a deep neural network (DNN).

Also, the pulse diagnosis apparatus 100 may generate pressure, may measure vibrations of a pulse according to the generated pressure, may draw a pulse waveform separate from a blood flow rate waveform through the measurement, and may diagnose the pulse by further considering the drawn pulse waveform. In addition, the pulse diagnosis apparatus 100 may perform a primary pulse diagnosis by using pulse models stored therein, and, when it is determined that there is a suspicious symptom as a result of the primary pulse diagnosis, may notify a user of the result through a display 10 and simultaneously derive final pulse diagnosis data by cooperating with a diagnosis server 300.

The diagnosis server 300 according to one embodiment of the present invention may be embodied as a web server, may identify a plurality of such pulse diagnosis apparatuses 100, and may store and manage data received from the pulse diagnosis apparatus 100. In addition, the pulse diagnosis apparatus 100 may learn using personalized data. Conversely, the diagnosis server 300 may learn using data of a plurality of users and may improve diagnosis performance through the learned data. Also, the diagnosis server 300 may periodically transmit pulse models embodied according to the learning to the pulse diagnosis apparatus 100 to support improvement of pulse diagnosis performance of the pulse diagnosis apparatus 100. The above-described diagnosis server 300 may have the same hardware configuration as that of a general web server or network server. However, the diagnosis server 300 may include a program module embodied through languages such as C, C++, java, visual basic, visual C, and the like as software.

Also, the pulse diagnosis apparatus 100 may derive pulse diagnosis data by totally considering medical examination data obtained through a question and answer process with a user or past diagnosis history data in addition to a waveform of the blood flow rate measured by the blood flow rate sensor. Here, when the pulse diagnosis apparatus 100 is configured to include an interface for interacting with the user, the pulse diagnosis data may be derived by directly receiving a response of the user and a determination may be performed by receiving an answer of the user from a connected terminal 200.

The terminal 200 according to one embodiment of the present invention may be connected to the pulse diagnosis apparatus 100 through a local area network (LAN), as described above, may support a variety of user inputs for diagnosing a pulse, or may provide a derived pulse diagnosis data to the user. For this, the terminal 200 may execute a program for transmitting and receiving information with the pulse diagnosis apparatus 100.

Additionally, a communication network 500 according to one embodiment of the present invention refers to a communication network which supports data transmission/reception between the pulse diagnosis apparatus 100 and the diagnosis server 300, and more particularly, may be formed by combining a variety of communication networks embodied by using a variety of wired/wireless communication technologies, such as an intra network, a mobile communication network, a satellite communication network, and the like rather than a single communication network. Also, the communication network 500 may include a cloud computing network which stores computing sources such as hardware, software, and the like and is able to provide a computing source needed by a client to a corresponding terminal. Here, cloud computing refers to a computing environment in which data is permanently stored in a server on the Internet and temporarily stored in a client terminal such as a desktop PC, a tablet computer, a laptop computer, a netbook computer, a smart phone, and the like. A cloud computing network refers to a computing-environment access network which stores all data of the user in a server on the Internet and allows the user to use the data anytime and anywhere through various IT devices. The above-described communication network 500 corresponds to concept which includes a network such as code division multiple access (CDMA), wideband CDMA (WCDMA), a global system for mobile communications (GSM), long term evolution (LTE) recently receiving attention, an evolved packet core (EPC), and the like in addition to a closed network such as a LAN, a wide area network (WAN), and the like and an open network such as the Internet as well as all next-generation networks and cloud computing networks which will be embodied in the future.

Hereinafter, an example of the pulse diagnosis apparatus 100 according to one embodiment of the present invention will be described.

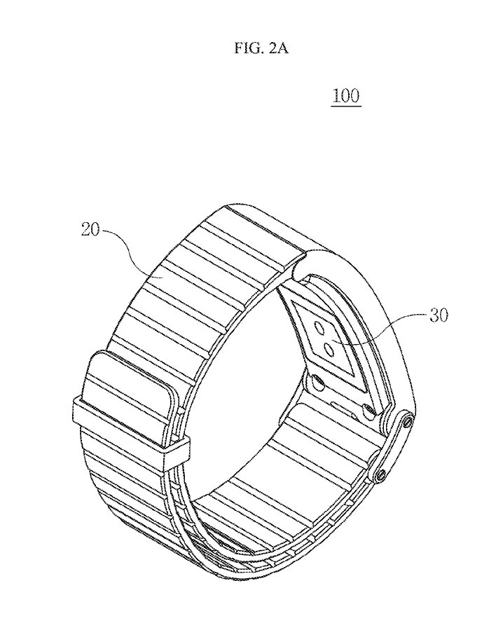

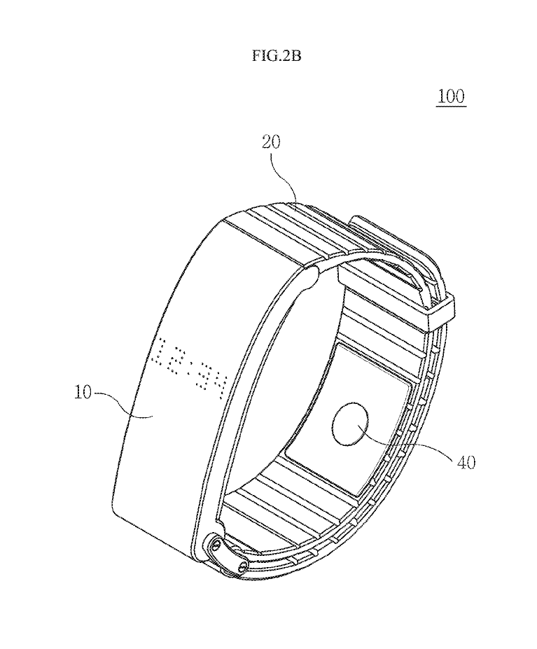

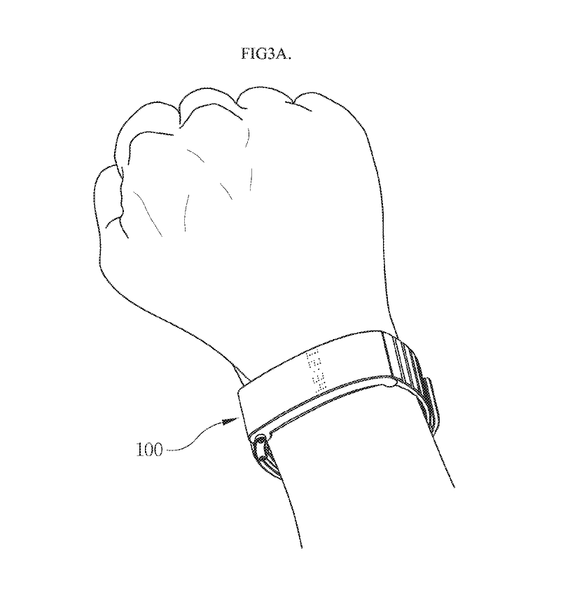

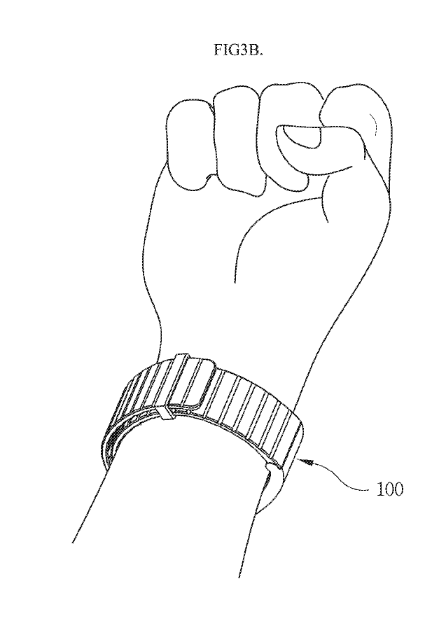

The pulse diagnosis apparatus 100 according to one embodiment of the present invention refers to an apparatus capable of coming into contact with a user's body part and measuring a wave of a pulse. The above-described pulse diagnosis apparatus 100 may be embodied as a smart watch, as shown in FIGS. 2A, 2B, 3A and 3B.

That is, FIGS. 2A and 2B are views illustrating an example of the pulse diagnosis apparatus according to one embodiment of the present invention, and FIGS. 3A and 3B are views illustrating a state in which the pulse diagnosis apparatus according to one embodiment of the present invention is worn on a part of the user's body.

As shown in FIGS. 2A, 2B, 3A and 3B, the pulse diagnosis apparatus 100 may be embodied as a smart watch worn on a wrist of the user, and the pulse diagnosis apparatus 100 may be embodied as one module in the smart watch.

Here, the smart watch according to one embodiment of the present invention, as shown in FIGS. 3A and 3B, includes a display 10 and may be worn on the wrist of the user by using a band 20 to allow the display 10 to be positioned on the back of the user's hand. Also, the pulse diagnosis apparatus 100, as shown in FIG. 2A, includes a blood flow rate sensor 30. The blood flow rate sensor 30 may be attached to a rear surface of the display 10 and may measure a blood flow rate change at a wrist area on the back of the user's hand by using the blood flow rate sensor 30.

Also, the pulse diagnosis apparatus 100, as shown in FIG. 2B, may include a pressure sensor 40. The pressure sensor 40 according to one embodiment may be installed on a rear surface opposite the blood flow rate sensor 30, as shown in the drawing. For example, when the blood flow rate sensor 30 is installed at a position of the display 10 of the smart watch, the pressure sensor 40 may be installed at a connection position of the band 20 opposite thereto. Conversely, when the blood flow rate sensor 30 is installed at the connection position of the band 20, the pressure sensor 40 may be installed at the position of the display 10 in an opposite direction thereto.

Accordingly, as shown in FIGS. 3A and 3B, when the blood flow rate sensor 30 is located at the display 10, the blood flow rate sensor 30 may be positioned at the wrist area on the back of the user's hand and may measure a blood flow rate change of blood which flows through the wrist area on the back of the user's hand. Also, the pressure sensor 40 may be positioned at the wrist area on the back of the user's hand and may apply pressure to the wrist area on the back of the user's hand. Here, the pressure sensor 40 may include a pressure generation module (not shown) which generates the pressure and a measurement module (not shown) which measures vibration of a pulse according to the additionally generated pressure. Here, the measurement module and the pressure generation module according to one embodiment of the present invention may be installed on the same rear surface and spaced at a certain interval apart.

Also, depending on an embodiment, the blood flow rate sensor 30 and the pressure sensor 40 may be positioned to be in the same direction and not to face each other. That is, the blood flow rate sensor 30 is located at the display 10, the pressure sensor 40 may be located at a band connection part opposite thereto or may be located at a position spaced at a certain interval apart from the same band surface as the blood flow rate sensor 30 located at the part of display 10.

The exemplary embodiment of the pulse diagnosis apparatus 100 according to one embodiment of the present invention has been described above. However, although the exemplary embodiment of the pulse diagnosis apparatus 100 has been described with reference to FIGS. 2A, 2B, 3A and 3B, the description is merely one example, and the pulse diagnosis apparatus 100 is not limited thereto. That is, the pulse diagnosis apparatus 100 may be embodied as various types of devices in addition to the smart watch. The above-described pulse diagnosis apparatus 100 may be embodied as any apparatus which is attached to a user's body part to measure a change of a pulse of the user. For example, the pulse of the user may be measured at various parts of the user such as the dorsalis pedis artery at a center of the top of a foot, a popliteal artery at the crook of a knee, an abdominal artery at the abdominal region, and the like in addition to the radial artery. Accordingly, it should be noted that the pulse diagnosis apparatus 100 may be embodied as any apparatus capable of being worn on a place of the user's body at which the pulse is located and measuring a blood flow rate waveform according to a change of the pulse.

Hereinafter, significant components and an operation method of the pulse diagnosis apparatus 100 according to one embodiment of the present invention will be described in more detail.

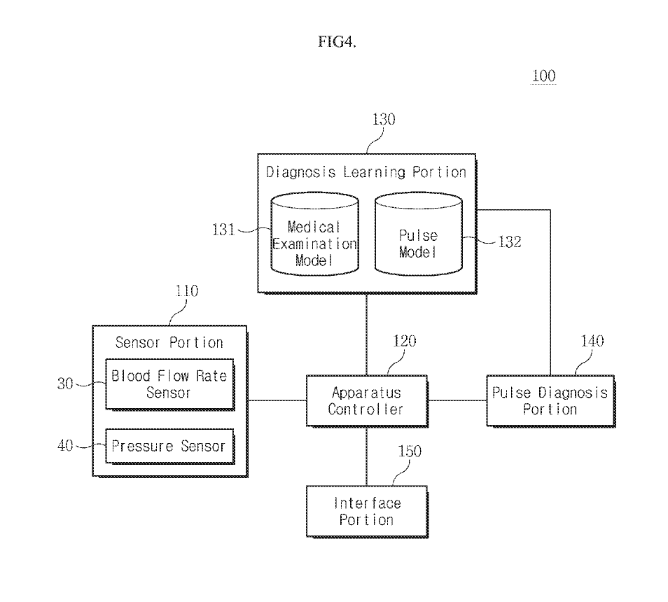

FIG. 4 is a configuration diagram illustrating significant components of the pulse diagnosis apparatus according to one embodiment of the present invention.

Referring to FIG. 4, the pulse diagnosis apparatus 100 may include a sensor portion 110, an apparatus controller 120, a diagnosis learning portion 130, a pulse diagnosis portion 140, and an interface portion 150.

Each component will be described in more detail. The sensor portion 110 may basically include the blood flow rate sensor 30. The blood flow rate sensor 30 is a sensor capable of measuring a blood flow rate change of a user and may be, for example, a photoplethysmograph sensor. When the blood flow rate sensor 30 is embodied as the photoplethysmograph sensor, the blood flow rate sensor 30 may include a light emitting diode (LED, not shown) and a light receiving element (not shown). Here, the LED may emit light toward a surface of the user's body, and the light receiving element may receive light transmitted toward the surface of user's skin and reflected and may convert the light into an electrical signal. To convert and output the electrical signal, the sensor portion 110 may further include an analog-digital (AD) conversion module (not shown).

Additionally, the blood flow rate sensor 30 may continuously emit light toward the user's body part and may sense and measure a level of the reflected light, and measurement data may be generated as a digital blood flow rate waveform and transmitted to the apparatus controller 120. Meanwhile, the blood flow rate sensor 30 is exemplary described as operating in an optical method but is not limited thereto. Any sensor which is capable of coming into contact with the body part and measuring a blood flow rate change may be applied as the blood flow rate sensor 30.

Meanwhile, depending on embodiments, the sensor portion 110 of the pulse diagnosis apparatus 100 according to one embodiment of the present invention may further include the pressure sensor 40.

When the sensor portion 110 includes the pressure sensor 40, the pressure sensor 40 may include the pressure generation module which generates pressure. Here, the pressure generation module may generate a certain level of pressure. The generated pressure may pressurize a part of the user's body (preferably, a pulsating part of a wrist), and a blood flow rate change occurs as the body part is pressurized. The blood flow rate change may be measured by the blood flow rate sensor 30.

Also, the pressure sensor 40 may further include the measurement module which measures a pulse level changed according to the generated pressure. That is, the pressure sensor 40 may generate pressure capable of pressurizing the part of the body and simultaneously sense and measure a pulse vibration changed according to a pressure change. Here, the pressure sensor 40 may generate a pulse waveform by converting the measured pulse vibration into a digital form.

In addition, when the sensor portion 110 includes the blood flow rate sensor 30 and the pressure sensor 40, the blood flow rate sensor 30 and the pressure sensor 40 may simultaneously operate. However, basically, only the blood flow rate sensor 30 may operate and the pressure sensor 40 may selectively operate. Here, the apparatus controller 120 may monitor the blood flow rate waveform measured by the blood flow rate sensor 30 and may control the pressure sensor 40 to generate pressure when a preset condition is sensed. Here, the preset condition may include a case in which a particular waveform is sensed by analyzing the blood flow rate waveform. The pressure may be generated by controlling the pressure sensor 40 at certain intervals regardless of the blood flow rate waveform according to the embodiment.

The apparatus controller 120 performs a function of totally controlling the pulse diagnosis apparatus 100 according to one embodiment of the present invention. Particularly, when a blood flow rate waveform is measured by the sensor portion 110, the apparatus controller 120 may transfer the blood flow rate waveform to the diagnosis learning portion 130 to control a process of deriving pulse data through a pulse model previously built into the diagnosis learning portion 130.

In addition, the apparatus controller 120 may monitor the blood flow rate waveform measured by the blood flow rate sensor 30 and may selectively control operation of the pressure sensor 40 depending on the monitored blood flow rate waveform when the sensor portion 110 includes the pressure sensor 40.

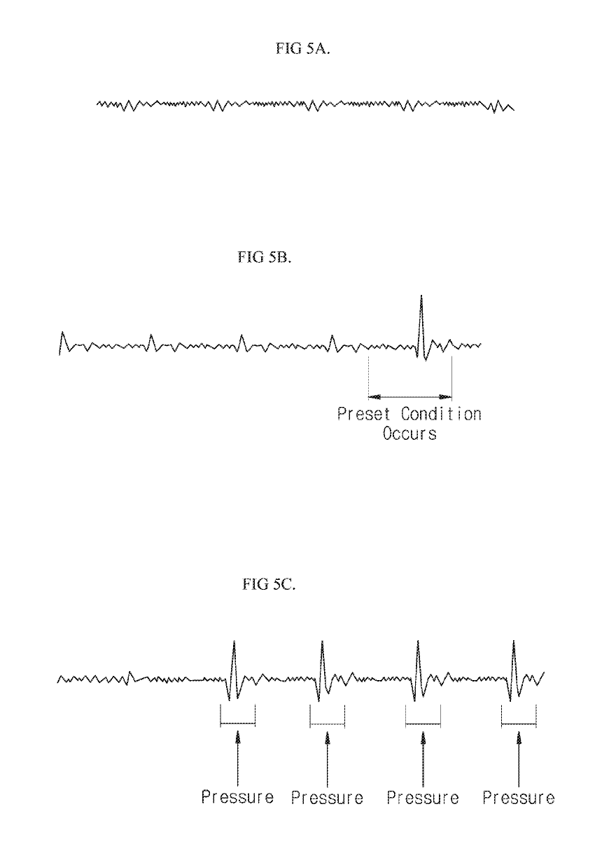

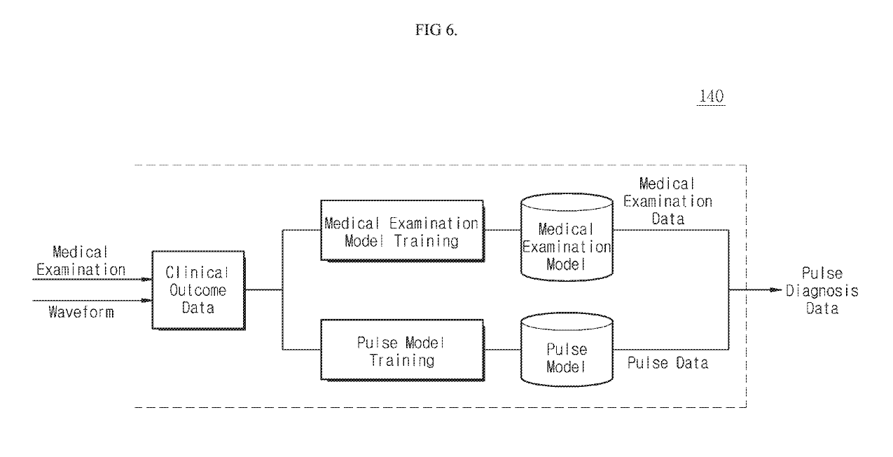

The process will be described in more detail. As shown in FIG. 5A, the apparatus controller 120 monitors a blood flow rate waveform measured by the blood flow rate sensor 30. Also, when a change of a certain level or more occurs in the waveform, as shown in FIG. 5B, the apparatus controller 120 may determine that the preset condition occurs. Also, the apparatus controller 120 controls the pressure sensor 40 to generate a certain level of pressure.

The pressure sensor 40 may generate the certain level of pressure according to the control of the apparatus controller 120, and the generated pressure may pressurize a body part such that a blood flow rate change may occur. Accordingly, whenever the pressure is applied, a change may occur in the blood flow rate waveform measured by the blood flow rate sensor 30, as shown in FIG. 5C.

In addition, when a change in level of the blood flow rate waveform measured by the blood flow rate sensor 30 is determined to be insignificant or is a certain level or less in a state in which the pressure sensor 40 is controlled to generate the certain level of pressure, the apparatus controller 120 may determine that the pressure is being incorrectly applied to the user's body and increase the level of the pressure generated by the pressure sensor 40 or may provide notification information to the user through the interface portion 150 to allow the user to adjust a position of the pulse diagnosis apparatus 100. That is, the pressure sensor 40 preferably comes into close contact with the wrist of the user and applies the pressure to a pulse generated at the radial artery. When the pressure sensor 40 applies the pressure to another position instead of a designated position, a function of notifying the user to adjust the pulse diagnosis apparatus 100 to the correct position may be performed.

Also, when a pulse waveform is measured by the sensor portion 110, the apparatus controller 120 may transfer the pulse waveform to the diagnosis learning portion 130 like the blood flow rate waveform.

Also, when medical examination data formed by interviewing a patient is present, the apparatus controller 120 may perform a function of transferring the medical examination data to the diagnosis learning portion 130. Here, the medical examination data may be medical examination data of the user input through the interface portion 150 of the pulse diagnosis apparatus 100 or may be medical examination data input through the terminal 200 of the user. Also, the apparatus controller 120 may check whether past diagnosis history data is present. When diagnosis history data is present, the diagnosis history data may be transferred with the medical examination data to the diagnosis learning portion 130.

The diagnosis learning portion 130 performs a function of deriving pulse data by applying a waveform transferred through the apparatus controller 120 to a pre-built pulse model 132. Here, the waveform may basically include a blood flow rate waveform, and may further include a pulse waveform.

Also, when basic data for a medical examination is input, the diagnosis learning portion 130 may derive the medical examination data by using the basic data.

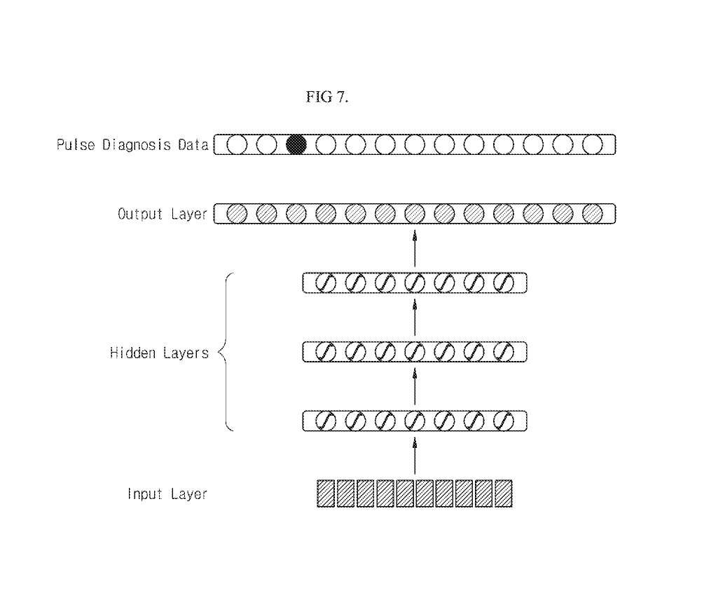

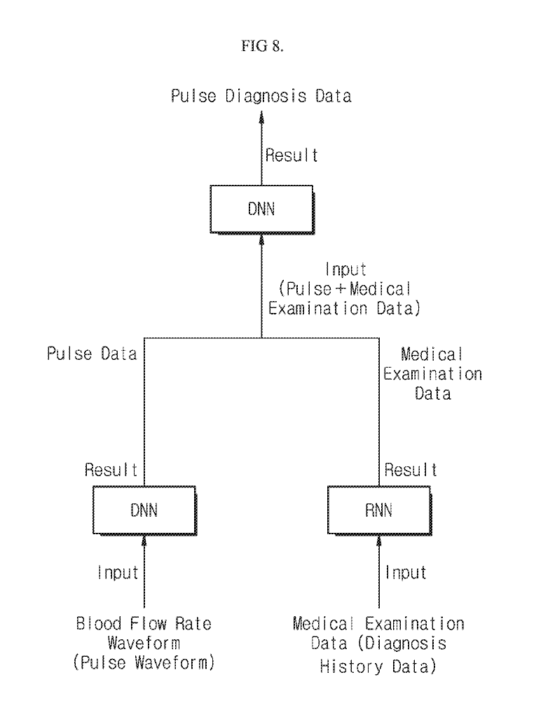

For this, first, the diagnosis learning portion 130 performs a process of learning and building a medical examination model and a pulse model. This process will be described with reference to FIGS. 6 and 7.

FIG. 6 is a view illustrating an example of a process of building a learning model according to one embodiment of the present invention, and FIG. 7 is a view illustrating an example of a DNN for building a learning model according to one embodiment of the present invention.

First, as shown in FIG. 6, the pulse diagnosis apparatus 100 may learn and build a medical examination model and a pulse model in advance to diagnose a pulse. That is, in a state in which clinical outcome data for a medical examination and a waveform is present, a medical examination model and a pulse model are built using the DNN. Here, as shown in FIG. 7, the DNN refers to a network formed of a multilayer perceptron structure which includes an input layer, an output layer, and a plurality of hidden layers hidden between the input layer and the output layer. Each of the layers may include a plurality of nodes corresponding to artificial neurons, and a connection among neurons of different layers may be determined by learning. Particularly, an output value at one node is determined to be an activation function output value of the node. Here, an input of an activation function may refer to the sum obtained by adding up all nodes connected to the node.

The pulse diagnosis apparatus 100 sets an input value (a histogram of frequency characteristics analyzed from a waveform of a measured blood flow rate and a change thereof, a level of pressure, and the like) of the input layer, sets an output value (pulse data) of the output layer, and performs learning through the hidden layer by using the DNN. The diagnosis learning portion 130 may build the pulse model 132 through the above-described process.

Meanwhile, the diagnosis learning portion 130 performs medical examination model training by using a recurrent neural network (RNN) and may accordingly build a medical examination model 131. Here, the RNN is a model used for extracting a time-serial correlation (or connection) among input data and analyzes and learns a time-serial correlation of text data such as the medical examination data and the diagnosis history data and builds the medical examination model 131.

As described above, in a state in which the medical examination model 131 and the pulse model 132 are built using clinical outcome data, the diagnosis learning portion 130 may derive pulse data by using the pulse model 132 when a blood flow rate waveform (or a pulse waveform) is transferred through the apparatus controller 120 and may derive medical examination data by using the medical examination model 131 when basic diagnosis data such as the medical examination data or the diagnosis history data is transferred.

Also, the derived pulse data and medical examination data may be transferred to the pulse diagnosis portion 140 such that the pulse diagnosis portion 140 may ultimately derive the pulse diagnosis data. Here, the pulse diagnosis portion 140 derives the final pulse diagnosis data through the DNN which sets the pulse data and the medical examination data to be an input value of the pulse diagnosis portion 140 and sets the pulse diagnosis data to be an output value thereof.

Additionally, the diagnosis learning portion 130 may continuously learn the medical examination model and the pulse model by using the pulse diagnosis data derived by the pulse diagnosis portion 140, may build a customized learning model based on personalized data according thereto, and may further increase performance through a user-customized diagnosis. Also, the diagnosis learning portion 130, as described with reference to FIG. 1, may periodically receive a medical examination model and a pulse model based on data of another user from the diagnosis server 300 and may train by learning models and further considering the received medical examination model and pulse model.

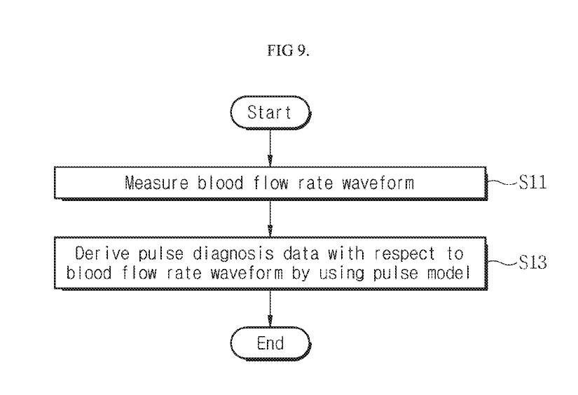

An overall process of deriving pulse diagnosis data by the pulse diagnosis apparatus 100 will be described with reference to FIG. 8.

FIG. 8 is a view illustrating a process of deriving pulse diagnosis data by using a neural network algorithm according to one embodiment of the present invention.

As shown in FIG. 8, the pulse diagnosis apparatus 100 draws a conclusion by applying a DNN which sets a blood flow rate waveform generated by the sensor portion 110 to be an input and sets pulse data to be a result. Here, the waveform set to be the input may include a pulse waveform in addition to a blood flow rate waveform. In this case, the waveforms may be merged and set to be the input. Also, all values like a pressure value 1, a blood flow rate waveform histogram, a pressure value 2, a blood flow rate waveform histogram, and the like may be converted into one vector and applied as the input.

Simultaneously, when medical examination data or diagnosis history data of a user is checked through the interface portion 150, the pulse diagnosis apparatus 100 may derive medical examination data, which is a result of applying the medical examination data or the diagnosis history data to an RNN.

Also, the pulse data derived through the DNN and the medical examination data derived through the RNN are set to be an input of the DNN and are applied to the DNN which sets pulse diagnosis data to be a result to derive final pulse diagnosis data.

The pulse diagnosis apparatus 100 performs a pulse diagnosis according to a neural network algorithm adequate for diagnosing a pulse such that the pulse diagnosis may be more accurately performed.

Referring back to FIG. 4, the interface portion 150 may transmit a variety of pieces of data for interaction with the user and a pulse diagnosis. For example, the interface portion 150 may include the display 10, as shown in FIG. 2B, may display various queries for generating the medical examination data through the display 10, generate the medical examination data by using answers of the user input according thereto, and transfer the generated medical examination data to the apparatus controller 120. Also, the interface portion 150 may include a communication module (not shown) and may receive a medical examination mode and a pulse model from the diagnosis server 300 and transmit the medical examination mode and the pulse model through the communication module. Also, it is possible to control an additional pulse diagnosis to be performed by the diagnosis server 300 by transferring the pulse diagnosis data derived by the pulse diagnosis portion 140 and a variety of pieces of data to the diagnosis server 300.

The significant components and operations of the pulse diagnosis apparatus 100 according to one embodiment of the present invention have been described above. A memory mounted in the pulse diagnosis apparatus 100 stores data in the apparatus. In one example, the memory is a computer-readable medium. In one example, the memory may be a volatile memory unit. In another example, the memory may be a nonvolatile memory unit. In one example, a storage device is a computer-readable medium. In various different examples, the storage device may include, for example, a hard disk drive, an optical disk device, and any other mass storage device.

In addition, the term “module” used in the embodiment refers to a software component which performs certain functions. For example, a “module” includes components such as software components, object-oriented software components, class components, and task components, processes, functions, properties, procedures, subroutines, segments of a program code, drivers, data, a database, data structures, tables, arrays, and variables. Also, functions provided in “components” and “modules” may be combined into a smaller number of “components” and “modules” or may be further divided into additional “components” and “modules”.

Although an exemplary apparatus configuration has been described in the specification and shown in the drawings, functional operations and concept described herein may be embodied as different types of digital electronic circuits, computer software, firmware, hardware, and a combination of one or more thereof, which include the structures disclosed above and structural equivalents. The embodiments of the concept described herein may be embodied as one or more computer program products, in other words, one or more modules related to a computer program instruction encoded in a tangible program storage medium to control operations of the apparatus according to one embodiment of the present invention or to execute according thereto. The computer-readable medium may be one of a machine-readable storage device, a machine-readable storage circuit board, a memory device, a machine-readable composition of materials which influence a radio wave signal, and a combination of one or more thereof.

Hereinafter, a pulse diagnosis method used in the pulse diagnosis system according to one embodiment of the present invention will be described.

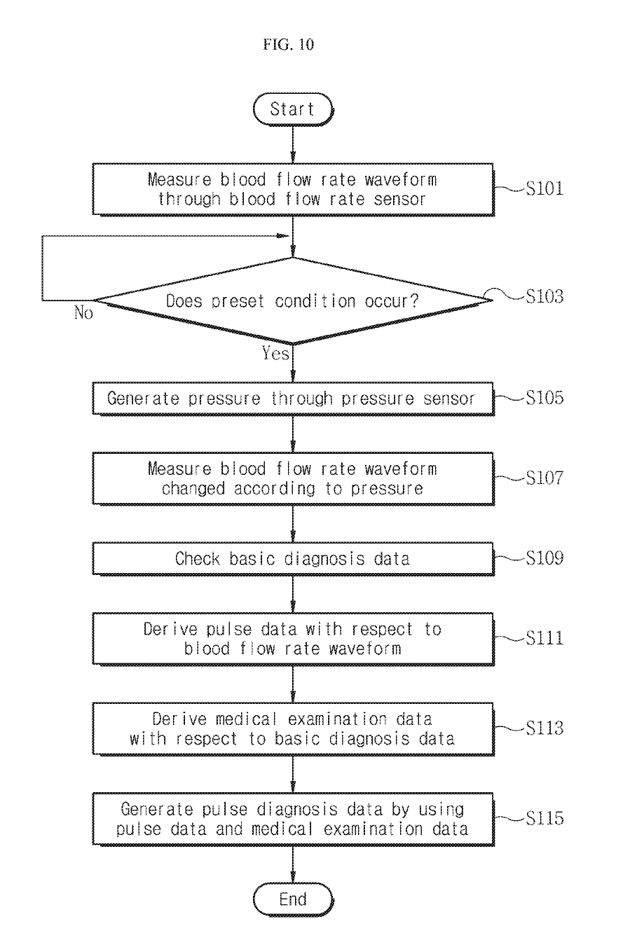

FIG. 9 is a schematic flowchart illustrating a pulse diagnosis method according to one embodiment of the present invention, and FIG. 10 is a more detailed flowchart illustrating the pulse diagnosis method according to one embodiment of the present invention.

As shown in FIG. 9, the pulse diagnosis apparatus 100 basically includes the blood flow rate sensor 30, measures and generates a blood flow rate waveform through the blood flow rate sensor 30 (S11), and derives pulse diagnosis data with respect to the blood flow rate waveform by using a pulse model generated through pulse model training by using a DNN (S13).

In addition, the pulse diagnosis apparatus 100 may include the pressure sensor 40. When the pressure sensor 40 is included in the pulse diagnosis apparatus 100, a blood flow rate waveform according to a pressure change may be measured and a pulse diagnosis may be performed according thereto.

A case in which the pulse diagnosis apparatus 100 includes the pressure sensor 40 will be described with reference to FIG. 10.

For reference, the pulse diagnosis apparatus 100 may be embodied as a smart watch and may continuously measure a blood flow rate waveform through the blood flow rate sensor 30 while being worn on a human body part, such as a wrist of a user, at which a change in arteries may be sensed (S101).

Also, the pulse diagnosis apparatus 100 may monitor the measured blood flow rate waveform and may sense that body conditions of the user are unusual when a waveform different from a usual blood flow rate waveform is sensed, as described with reference to FIG. 5. Here, to more accurately determine conditions of the user, the pulse diagnosis apparatus 100 drives the pressure sensor 40 and generates a certain level of pressure (S105).

Also, the pulse diagnosis apparatus 100 measures and generates a blood flow rate waveform changed according to the generated pressure (S107). Here, when a degree of change in the blood flow rate waveform according to the generated pressure is insignificant, the pulse diagnosis apparatus 100 may control the pressure sensor 40 to generate a higher pressure. Also, when the degree of change in the blood flow rate waveform is determined as being insignificant even when the higher pressure is generated, the pulse diagnosis apparatus 100 may notify the user to adjust a position of the pulse diagnosis apparatus 100.

In addition, the pulse diagnosis apparatus 100 checks basic diagnosis data (S109). Here, the basic diagnosis data may include medical examination data obtained through a question and answer process. For example, the pulse diagnosis apparatus 100 may ask a question for checking the conditions of the user such as “Have you suffered from a cold within the past five days?” and may obtain the medical examination data through an answer according thereto. Also, the basic diagnosis data may include past diagnosis history data.

Afterward, the pulse diagnosis apparatus 100 derives pulse data by using the blood flow rate waveform (S111) and derives the medical examination data by using the basic diagnosis data (S113).

Next, the pulse diagnosis apparatus 100 derives pulse diagnosis data by using the pulse data and the medical examination data (S115).

Since the pulse diagnosis apparatus 100 is embodied in a wearable form worn on a user's body part, a diagnosis may be performed by continuously monitoring conditions of the user.

Also, the pulse diagnosis apparatus 100 may check a pulse state of the user by using a blood flow rate waveform and may derive more precise pulse diagnosis data by additionally totally considering a variety of pieces of data such as a pulse waveform, medical examination data, and the like.

Also, the pulse diagnosis apparatus 100 derives pulse data through the DNN, derives medical examination data through the RNN, and then derives final pulse diagnosis data through the DNN such that a more precise pulse diagnosis may be performed by applying a neural network algorithm optimized for the pulse diagnosis.

The pulse diagnosis method according to one embodiment of the present invention has been described above.

The above-described pulse diagnosis method according to one embodiment of the present invention may be provided as a computer-readable medium adequate for storing a computer program instruction and data. A program recorded in a recording medium for performing the pulse diagnosis method according to one embodiment of the present invention may include generating a blood flow rate waveform through a blood flow rate sensor capable of measuring a blood flow rate change and deriving pulse data with respect to the measured blood flow rate waveform by applying the blood flow rate waveform to a pulse model generated by using a DNN.

Here, the program recorded in the recording medium may be read by, installed in, and executed by a computer to perform the above-described functions.

Here, to allow the computer to read the program recorded in the recording medium and execute the functions embodied in the program, the above-described program may include codes coded in computer languages such as python in addition to C, C++, java, and the like, which are readable by a central processing unit (CPU) of the computer through an apparatus interface of the computer.

These codes may include function codes related to functions and the like which define the above-described functions, and may include control codes related to an execution procedure necessary for the CPU of the computer to execute the above-described functions according to a certain procedure. Also, these codes may further include memory reference-related codes which refer to a position (address) in the computer or an external memory for obtaining additional information or a medium necessary for the CPU of the computer to execute the above-described functions. Also, when it is necessary to communicate with any other remote computer, server, or the like to allow the CPU of the computer to execute the above-described functions, the codes may further include communications-related codes with respect to how the CPU of the computer can communicate with the other remote computer, server, or the like for using a communication module, which information or medium is to be transmitted and received during the communication, and the like.

A computer-readable medium adequate for storing the above-described computer program instruction and data, for example, a recording medium includes a magnetic medium such as a hard disk drive (HDD), a floppy disk, and a magnetic tape, an optical recording medium such as a CD-ROM and digital video disk (DVD), a magneto-optical medium such as a floptical disk, and a semiconductor memory such as a ROM, a RAM, a flash memory, an EPROM, and an electrically EPROM (EEPROM). The CPU and the memory may be supplemented by or integrated with a special-purpose logic circuit.

In addition, the computer-readable recording medium may be distributed to computer systems over a network such that computer-readable codes may be stored and executed in a distributed manner Also, functional programs for embodying the present invention and codes, code segments, and the like related thereto may be easily deduced and changed by programmers of ordinary skill in the art in consideration of a system environment and the like of a computer which reads the recording medium and executes the program.

Although the specification includes details of a plurality of particular embodiments, the embodiments are not to be understood as limiting the scope of the present invention, rather, the claims should be understood as a description on features characterized by the particular embodiments of the present invention. Particular features described in the context of separate embodiments in the specification may be embodied as being combined in a single embodiment. Conversely, a variety of features described in the context of a single embodiment may also be embodied separately or as adequate sub-combinations with a plurality of embodiments. In addition, although features may operate in a particular combination and are described as initially claimed, one or more features from the claimed combination may be excluded from the combination in some cases, and the claimed combination may be changed into a sub-combination or a modification of the sub-combination.

Likewise, although operations are illustrated in a particular order in the drawings, the operations are not to be understood as necessarily being performed in the particular order or a sequential order to obtain a preferable result or to perform all of the illustrated operations. In a particular case, multitasking and parallel processing may be advantageous. Also, it is not necessary for a variety of system components of the above-described embodiment to be separated for all embodiments. It should be understood that the above-described program components and systems may be generally integrated to be a single software product or packaged in a multiple software product.

What is claimed is:

1. A pulse diagnosis apparatus comprising:
a sensor portion which comprises a blood flow rate sensor capable of measuring a blood flow rate change and generates a blood flow rate waveform through the blood flow rate sensor;
an apparatus controller which controls an application of the generated blood flow rate waveform to a pulse model generated using a deep neural network (DNN); and
a pulse diagnosis portion which derives pulse diagnosis data with respect to the blood flow rate waveform by using the pulse model.

2. The pulse diagnosis apparatus of claim 1, wherein the sensor portion further comprises a pressure sensor, and
the apparatus controller controls the pressure sensor to generate a certain level of pressure of a certain level when a preset condition occurs.

3. The pulse diagnosis apparatus of claim 2, wherein the apparatus controller controls an application of a blood flow rate waveform changed according to a pressure change generated by the pressure sensor and a pressure value generated by the pressure sensor to the pulse model.

4. The pulse diagnosis apparatus of claim 2, wherein when the pressure sensor comprises a pressure generation module which generates the pressure and a measurement module which measures a vibration of a pulse according to the pressure generated by the pressure generation module and generates a pulse waveform, and the apparatus controller controls an application of a blood flow rate waveform changed according to a pressure change generated by the pressure generation module and a pulse waveform generated by the measurement module to the pulse model.

5. The pulse diagnosis apparatus of claim 4, wherein the apparatus controller monitors the blood flow rate waveform changed according to the pressure change or checks a level of the vibration measured by the measurement module to adjust a position to which the pressure is applied.

6. The pulse diagnosis apparatus of claim 1, further comprising a diagnosis learning portion which builds the pulse model through pulse model training using a DNN based on previously obtained clinical outcome data and builds a medical examination model through medical examination model training using a recurrent neural network (RNN).

7. The pulse diagnosis apparatus of claim 6, wherein when pulse data is derived through the pulse model and medical examination data is derived through the medical examination model, the pulse diagnosis portion derives pulse diagnosis data through a DNN which sets the pulse data and the medical examination data to be an input value and sets the pulse diagnosis data to be an output value.

8. A pulse diagnosis apparatus comprising:
a sensor portion which comprises a blood flow rate sensor capable of measuring a blood flow rate change and generates a blood flow rate waveform through the blood flow rate sensor;
a diagnosis learning portion which learns and generates a pulse model by applying the generated blood flow rate waveform set to be an input and pulse data set to be an output to a DNN; and
a pulse diagnosis portion which performs a pulse diagnosis with respect to a blood flow rate waveform periodically measured through the sensor portion by applying the blood flow rate waveform to the pulse model.

9. The pulse diagnosis apparatus of claim 8, wherein the diagnosis learning portion learns and generates a medical examination model through medical examination model training by applying basic diagnosis data comprising at least one of medical examination data generated through a question and answer of a user and prestored diagnosis history data to an RNN, and
the pulse diagnosis portion derives pulse diagnosis data by applying pulse data derived through the pulse model and the medical examination data derived through the medical examination model to the DNN as an input thereof.

10. A pulse diagnosis method comprising:
generating a blood flow rate waveform through a blood flow rate sensor capable of measuring a blood flow rate change; and
deriving, by a pulse diagnosis apparatus, pulse data with respect to the measured blood flow rate waveform by applying the blood flow rate waveform to a pulse model generated using a DNN.

11. The pulse diagnosis method of claim 10, wherein when the pulse diagnosis apparatus further comprises a pressure sensor, the generating of the blood flow rate waveform comprises:
monitoring the measured blood flow rate waveform;
controlling the pressure sensor to generate a certain level of pressure when it is determined that a preset condition occurs as a result of the monitoring; and
continuously measuring and generating a blood flow rate waveform changed according to a pressure change.

12. The pulse diagnosis method of claim 11, comprising, when the pressure sensor comprises a pressure generation module which generates the pressure and a measurement module which measures a vibration of a pulse according to the pressure generated by the pressure generation module and generates a pulse waveform, generating a blood flow rate waveform changed according to the pressure change and the pulse waveform by using the measurement module.

13. The pulse diagnosis method of claim 10, further comprising, when basic diagnosis data which comprises at least one of medical examination data generated through a question and answer of a user and prestored diagnosis history data is checked, deriving medical examination data with respect to the basic diagnosis data by applying the basic diagnosis data to a medical examination model generated using an RNN.

14. The pulse diagnosis method of claim 13, further comprising, after the deriving of the medical examination data, deriving pulse diagnosis data through an RNN which sets the pulse data and the medical examination data to be an input value and sets the pulse diagnosis data to be an output value.
